(19) United States
(12) Patent Application Publication
(10) Pub. No.: US 2015/0140010 A1
(43) Pub. Date: May 21, 2015

(54) METHODS FOR DIAGNOSING AND TREATING FOCAL SEGMENTAL GLOMERULOSCLEROSIS

(71) Applicants: INSERM (INSTITUT NATIONAL DE LA SANTE ET DE LA RECHERCHE MEDICALE, Paris (FR); UNIVERSITE PARIS-SUD XI, Orsay Cedex (FR); ASSISTANCE PUBLIQUE - HOPITAUX DE PARIS, Paris (FR)

(72) Inventors: Antoine Durrbach, Villejuif Cedex (FR); Hanz Lorenzo, Villejuif Cedex (FR); Severine Beaudreuil, Villejuif Cedex (FR)

(73) Assignees: INSERM 9INSTITUT NATIONAL DE LA SANTE ET DE LA R- ECHERCHE MEDICALE), Paris (FR); UNIVERSITE PARIS-SUD XI, Orsay Cedex (FR); ASSISTANCE PUBLIQUE-HOPITAUX DE PARIS, Paris (FR)

(21) Appl. No.: 14/402,539

(22) PCT Filed: May 22, 2013
(86) PCT No.: PCT/EP2013/060449

§ 371 Date: Nov. 20, 2014

(30) Foreign Application Priority Data
May 22, 2012 (EP) 12305563.4

(51) Int. Cl. C12Q 1/48; C12N 15/113; A61K 38/17; C07K 16/28; C07K 16/40

(52) U.S. Cl. CPC C12Q 1/485; C07K 16/2839; C07K 16/40; A61K 38/1777; C12N 15/1138; C07K 2317/76; C12N 2310/11; C12N 2310/14; C12N 2310/12; G01N 2333/91205; G01N 2800/347; G01N 2800/50; G01N 2800/52; G01N 2333/70596; G01N 2500/04
USPC 424/158.1; 424/130.1; 514/20.9; 514/44 A; 514/44 R; 435/15; 435/7.21; 435/7.8

(57) ABSTRACT

The present invention relates to methods for diagnosing and treating focal segmental glomerulosclerosis. More particularly, the present invention relates to a method for determining whether a subject is at risk of having or developing a focal segmental glomerulosclerosis (FSGS) comprising the step consisting of determining the level of calcium/calmodulin-dependent serine protein kinase (CASK) in a blood sample obtained from the subject. The present invention also relates to an agent effective to inhibit the binding of CASK to hCD98 present on the surface of podocytes for use in the prevention or treatment of focal segmental glomerulosclerosis (FSGS) in a subject in need thereof.

### FIELD OF THE INVENTION

The present invention relates to methods for diagnosing and treating focal segmental glomerulosclerosis.

### BACKGROUND OF THE INVENTION

Focal segmental glomerulosclerosis (FSGS) accounts for 20% of all cases of glomerulonephritis, both in children and adults. Its main etiology is idiopathic but it may be due result of numerous processes (obesity, vesicourethral reflux, HIV infection, etc) or any situation of kidney hyperfiltration. It may also be associated with inherited disease resulted from mutations of podocyte molecules such as Nephrin or Podocyne. In some case, the disease can reappear very rapidly after renal transplantation suggesting that FSGS could be a systemic disease. In addition, its occurrence or relapse after vaccination, viral infection as well as it sensitivity to immunosuppressive drugs, has lead to the hypothesis that FSGS may corresponded to an abnormal regulation of lymphocyte functions.

Primary FSGS is associated with proteinuria frequently related with nephrotic syndrome. Its cortico-resistant form is the main manifestation of this disease leading to a poor prognosis. Most FSGS subjects progress to end-stage renal insufficiency within 3 to 7 years. FSGS is particularly serious in subjects after renal transplantation. This procedure is threatened by its high recurrence rate in the transplanted graft, which runs at 30% to 50% for the first transplant and up to 90% for the second, manifesting as nephrotic proteinuria.

Recent advances in renal physiology have shown that podocytes and the slit diaphragm between podocytes play an import role in establishing the filtration barrier to the leakage of proteins into the urine. Slit diaphragms are specialized cell adhesion structures found in the glomerular epithelium (podocytes), where they attach the adjoining foot processes to one another and are essential for glomerular filtration. In nephrotic syndrome, the foot process architecture of mature podocytes is lost, and the slit diaphragms are replaced by tight junctions comparables to those found in developing glomeruli.

Previous observations on the immediate recurrence after transplantation have proposed the presence of a circulating factor that alters the permeability of the glomerular filtration barrier, resulting in the reappearance of proteinuria. Interestingly, plasmapheresis and immunoabsortion on protein A column have been reported to be effective in removing this unknown factor, in reducing proteinuria, and improving the graft survival of the recurrence of FSGS. Indeed, these procedures are considered as the most effective treatments for subject affected of FSGS, although their prognosis is always poor. In light of the foregoing, a need in the art exists for indentifying the circulating factor liable to be responsible for FSGS so as to envisage new tools for the diagnosis and treatment of such a disease.

CASK is a membrane-associated guanylate kinase (MAGUK) containing multi-domain modules that mediate protein-protein interactions important for the establishment and maintenance of podocyte cell polarization. It is composed of a calmodulin-dependent protein kinase-like domain followed by PDZ, SH3, and guanylate kinase-like domains. Although it is ubiquitously localized, CASK is mainly expressed at neuronal synapses, lymphocytes as well as in renal podocytes. As a binding partner of nephrin, CASK participates in the maintenance of polarized podocytes architecture by linking membrane proteins and signaling molecules to the actin cytoskeleton and nucleus (Lehtonen S, Lehtonen E, Kudlicka K, Holthöfer H, Farquhar M G. “Nephrin forms a complex with adherens junction proteins and CASK in podocytes and in Madin-Darby canine kidney cells expressing nephrin”. Am J Pathol. 165 (3):923-36. 2004).

Initially human CASK (hCASK) has been localized at the intracellular side of the cell membrane. Nevertheless, recently it has been reported that hCASK binds extracellular molecules in Caco2-BBE intestinal cells. In addition, it has been demonstrated that hhCD98 and hCASK co-precipitate and co-localize both in vitro and in vivo, and that the PDZ-binding domain of hhCD98 is directly involved in this interaction. Moreover, it has been identified an ectokinase function on the surface of neutrophils which is capable to interact with the basolateral domain of intestinal epithelial cells. At this level, this activity it has been suggested as modulator of the hhCD98/hCASK interaction (Yan Y, Vasudevan S, Nguyen H, Bork U, Sitaraman S, Merlin D. “Extracellular interaction between hhCD98 and the PDZ class II domain of hCASK in intestinal epithelia”. J Membr Biol. 215 (1):15-26. 2007).

However up to now, the involvement of CASK in the development of focal segmental glomerulosclerosis has not yet been investigated.

### SUMMARY OF THE INVENTION

The present invention relates to a method for determining whether a subject is at risk of having or developing a focal segmental glomerulosclerosis (FSGS) comprising the step consisting of determining the level of calcium/calmodulin-dependent serine protein kinase (CASK) in a blood sample obtained from the subject.

In a particular embodiment, the methods of the invention further comprise a step consisting of comparing the determined level of CASK in the blood sample obtained from the subject with a reference level, wherein a difference between said determined level and said reference level is indicative whether said subject is at risk of having or developing a FSGS or is at risk of recurrence of FSGS after renal transplantation.

The present invention also relates to a method for determining whether a subject is at risk of recurrence of FSGS after renal transplantation comprising the step consisting of determining the level of calcium/calmodulin-dependent serine protein kinase (CASK) in a blood sample obtained from the subject.

A further aspect of the invention also relates to a method for determining the responsiveness of a subject suffering from a focal segmental glomerulosclerosis (FSGS) to a treatment comprising the step consisting of determining the level of calcium/calmodulin-dependent serine protein kinase (CASK) in a blood sample obtained from the subject.

The present invention relates to an agent effective to inhibit the binding of CASK to hCD98 present on the surface of podocytes for use in the prevention or treatment of focal segmental glomerulosclerosis (FSGS) in a subject in need thereof.

### DETAILED DESCRIPTION OF THE INVENTION

The inventors have now identified the circulating factor responsible for FSGS. They have indeed analyzed molecules associated with protein A columns in subjects having an early recurrence of FSGS after renal transplantation. They have identified by mass spectroscopy the presence of calcium/calmodulin-dependent serine protein kinase (CASK) that was found in a protein A column of subjects treated for recurrent FSGS after renal transplantation but not for those treated for auto-antibody mediated diseases. In addition, they have shown later that this molecule is able to induce structural changes of podocytes in vitro. Accordingly, CASK represents the soluble factor responsible of FSGS and the present invention relates to method for diagnosing and treating FSGS based on this finding.

Diagnostic Methods of the Invention:

The present invention relates to a method for determining whether a subject is at risk of having or developing a focal segmental glomerulosclerosis (FSGS) comprising the step consisting of determining the level of calcium/calmodulin-dependent serine protein kinase (CASK) in a blood sample obtained from the subject.

A “subject” in the context of the present invention is a human (male or female). Typically said subject has been previously diagnosed with heavy proteinuria.

By “blood sample” is meant a volume of whole blood or fraction thereof, eg, serum, plasma, etc.

“Risk” in the context of the present invention, relates to the probability that an event will occur over a specific time period, as in the conversion to FSGS, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.

Methods for determining the level of a biomarker protein such as CASK in a blood sample are well known in the art.

In a particular embodiment, the methods of the invention comprise contacting the blood sample with a binding partner capable of selectively interacting with the biomarker protein present in the blood sample. The binding partner may be an antibody that may be polyclonal or monoclonal, preferably monoclonal. In another embodiment, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985).

Alternatively, techniques described for the production of single chain antibodies (see e.g. U.S. Pat. No. 4,946,778) can be adapted to produce anti-CASK, single chain antibodies. Antibodies useful in practicing the present invention also include anti-CASK fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to CASK. For example, phage display of antibodies may be used. In such a method, single-chain Fv (scFv) or Fab fragments are expressed on the surface of a suitable bacteriophage, e. g., M13. Briefly, spleen cells of a suitable host, e. g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay.

Polyclonal and monoclonal antibodies for CASK are described, for example, in J. Neurosci., Apr. 15, 1996, 76 (8):2488-2494; J. Cell Biol. 142 (1):129-138, (1998). Cell 94:773-782, (1998). Cell 94:761-771, (1998), J Neurosci 22 (14):6092-6105, 2002, J Biol Chem 278 (11):9778-9783, 2003 and J. Neuroscience (2002) 22:6426-6436.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. 1997. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

In another embodiment, the binding partner may be a polypeptide that is able to bind to CASK. For example said polypeptide may comprise all or a portion of the extracellular domains of hCD98. Typically, said functional equivalents may comprise the class II PDZ-binding domain of hCD98 or a portion thereof.

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188.

The afore mentioned assays generally involve the binding of the binding partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

The level of biomarker protein may be measured by using standard immunodiagnostic techniques, including immunoassays such as competition, direct reaction, or sandwich type assays. Such assays include, but are not limited to, agglutination tests; enzyme-labelled and mediated immunoassays, such as ELISAs; biotin/avidin type assays; radioimmunoassays; immunoelectrophoresis; immunoprecipitation.

More particularly, an ELISA method can be used, wherein the wells of a microtiter plate are coated with a set of antibodies which recognize said biomarker protein. A blood sample containing or suspected of containing said biomarker protein is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Measuring the level of the biomarker protein (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said biomarker protein may be identified based on the known “separation profile” e. g., retention time, for that compound and measured using standard techniques.

Alternatively, the separated compounds may be detected and measured by, for example, a mass spectrometer.

The present invention also relates to a method for determining whether a subject is at risk of recurrence of FSGS after renal transplantation comprising the step consisting of determining the level of calcium/calmodulin-dependent serine protein kinase (CASK) in a blood sample obtained from the subject.

In a particular embodiment, the methods of the invention further comprise a step consisting of comparing the determined level of CASK in the blood sample obtained from the subject with a reference level, wherein a difference between said determined level and said reference level is indicative whether said subject is at risk of having or developing a FSGS or is at risk of recurrence of FSGS after renal transplantation.

In one embodiment, the reference values may be index values or may be derived from one or more risk prediction algorithms or computed indices for FSGS. A reference value can be relative to a number or value derived from population studies, including without limitation, such subjects having similar body mass index, total cholesterol levels, LDL/HDL levels, systolic or diastolic blood pressure, proteinuria, subjects of the same or similar age range, subjects in the same or similar ethnic group, or subjects having heavy proteinuria or nephrotic syndrome from other origin than FSGS.

In one embodiment of the present invention, the reference value is derived from the level of CASK in a control sample derived from one or more subjects who are substantially healthy. Such subjects who are substantially healthy lack traditional risk factors for FSGS: for example, those subjects have a serum cholesterol level less than 200 mg/dl, systolic blood pressure less than or equal to 140 mm Hg, diastolic blood pressure less than or equal to 85 mm Hg, non-current smoker, no history of proteinuria. In another embodiment, such subjects are monitored and/or periodically retested for a diagnostically relevant period of time (“longitudinal studies”) following such test to verify continued absence of FSG. Such period of time may be one year, two years, two to five years, five years, five to ten years, ten years, or ten or more years from the initial testing date for determination of the reference value. Furthermore, retrospective measurement of CASK levels in properly banked historical subject samples may be used in establishing these reference values, thus shortening the study time required, presuming the subjects have been appropriately followed during the intervening period through the intended horizon of the product claim.

Typically, the levels of CASK in a subject who is at risk for FSGS is deemed to be higher than the reference value obtained from the general population or from healthy subjects or from subjects who have developed nephrotic syndrome not related to FSGS or minimal change disease (MCD) such as diabetes mellitus nephropathies, amyloidosis or membranous nephropathies.

A further aspect of the invention also relates to a method for determining the responsiveness of a subject suffering from a focal segmental glomerulosclerosis (FSGS) to a treatment comprising the step consisting of determining the level of calcium/calmodulin-dependent serine protein kinase (CASK) in a blood sample obtained from the subject.

Typically said treatment may consist in administration of immunosuppressive drugs, biotherapies (steroids, calcineurin inhibitor, or agents as described after).

By “determining the responsiveness” of a subject to a treatment is meant evaluating the resolution or improvement of abnormal clinical features. For example, in a subject suffering from focal segmental glomerulosclerosis (FSGS) that responds to a treatment, a restoration of normal proteinuria can be observed. More specifically, “determining the responsiveness” of a subject to a treatment includes determining whether upon said treatment, the subject undergoes a complete remission, a partial remission, a remission with a high or a low risk of relapse, or whether said treatment will have no significant effect on the abnormal clinical features and/or the evolution of the disease.

In a particular embodiment, the method as above described further comprises the step of comparing the determined level of CASK in the blood sample obtained from the subject with a reference level, wherein a difference between said determined level and said reference level is indicative whether said subject responds to the treatment. Typically, the reference value is derived from the level of CASK in a control sample derived from one or more subjects who have responded or not responded to said treatment.

Therapeutic Methods of the Invention:

The present invention relates to an agent effective to inhibit the binding of CASK to a receptor present in the podocyte. More particularly the present invention relates to an agent effective to inhibit the binding of CASK thCD98 present on the surface of podocytes for use in the prevention or treatment of focal segmental glomerulosclerosis (FSGS) in a subject in need thereof.

As used herein, the term “treatment” refers to inhibiting the disease or condition, i.e. arresting its development; relieving the disease or condition, i.e. causing regression of the condition; or relieving the conditions caused by the disease, i.e. symptoms of the disease.

As used herein, the term “prevention” refers to preventing the disease or condition from occurring in a subject which has not yet been diagnosed as having it

As used herein, the term “hCD98” has its general meaning in the art and refers to the glycoprotein associated integrin formed by covalent linkage of CD98 heavy chain with several light chain (Chillaron J. et al. Am. J. Physiol. 281:995s1018.).

Typically, the agent includes but is not limited to an antibody, a small organic molecule, a polypeptide and an aptamer.

In one embodiment, the agent is an antibody. The invention embraces antibodies or fragments of antibodies having the ability to block the interaction between CASK and hCD98. The antibodies may have specificity to CASK or hCD98. In one embodiment, the antibodies or fragment of antibodies are directed to all or a portion of the extracellular domain of hCD98. In one embodiment, the antibodies or fragment of antibodies are directed to an extracellular domain of hCD98. More particularly this invention provides an antibody or portion thereof capable of inhibiting binding of hCD98 to CASK, which antibody binds to an epitope located within a region of hCD98, which region of hCD98 binds to CASK. Typically said epitope is located in the class II PDZ-binding domain of hCD98 (Yan Y, Vasudevan S, Nguyen H, Bork U, Sitaraman S, Merlin D. “Extracellular interaction between hhCD98 and the PDZ class II domain of hCASK in intestinal epithelia”. J Membr Biol. 215 (1):15-26. 2007). This invention also provides an antibody or portion thereof capable of inhibiting binding of hCD98 to CASK, which antibody binds to an epitope located within a region of CASK, which region of CASK binds to hCD98. Typically said epitope is located in the PDZ class II domain of CASK (Yan Y, Vasudevan S, Nguyen H, Bork U, Sitaraman S, Merlin D. “Extracellular interaction between hhCD98 and the PDZ class II domain of hCASK in intestinal epithelia”. J Membr Biol. 215 (1):15-26. 2007).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of CASK, or hCD98. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant CASK may be provided by expression with recombinant cell lines. hCD98 may be provided in the form of human cells expressing hCD98 at their surface. Recombinant forms of hCD98 or CASK may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al.,/. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In one embodiment of the agents described herein, the agent is a polypeptide. In a particular embodiment the polypeptide is a functional equivalent of hCD98. As used herein, a “functional equivalent of hCD98 is a compound which is capable of binding to CASK, thereby preventing its interaction with hCD98. The term “functional equivalent” includes fragments, mutants, and muteins of hCD98. The term “functionally equivalent” thus includes any equivalent of hCD98 obtained by altering the amino acid sequence, for example by one or more amino acid deletions, substitutions or additions such that the protein analogue retains the ability to bind to CASK. Amino acid substitutions may be made, for example, by point mutation of the DNA encoding the amino acid sequence.

Functional equivalents include molecules that bind CASK and comprise all or a portion of the extracellular domains of hCD98. Typically, said functional equivalents may comprise the class II PDZ-binding domain of hCD98 or a portion thereof.

The functional equivalents include soluble forms of the hCD98. A suitable soluble form of these proteins, or functional equivalents thereof, might comprise, for example, a truncated form of the protein from which the transmembrane domain has been removed by chemical, proteolytic or recombinant methods.

Preferably, the functional equivalent is at least 80% homologous to the corresponding protein. In a preferred embodiment, the functional equivalent is at least 90% homologous as assessed by any conventional analysis algorithm such as for example, the Pileup sequence analysis software (Program Manual for the Wisconsin Package, 1996).

The term “a functionally equivalent fragment” as used herein also may mean any fragment or assembly of fragments of hCD98 that binds to CASK. Accordingly the present invention provides a polypeptide capable of inhibiting binding of hCD98 to CASK, which polypeptide comprises consecutive amino acids having a sequence which corresponds to the sequence of at least a portion of an extracellular domain of hCD98, which portion binds to CASK. In one embodiment, the polypeptide corresponds to an extracellular domain of hCD98. In another one embodiment, the polypeptide corresponds to the class II PDZ-binding hCD98. In another one embodiment, the polypeptide corresponds to a portion of the class II PDZ binding domain of hCD98.

Functionally equivalent fragments may belong to the same protein family as the human hCD98 identified herein. By “protein family” is meant a group of proteins that share a common function and exhibit common sequence homology. Homologous proteins may be derived from non-human species. Preferably, the homology between functionally equivalent protein sequences is at least 25% across the whole of amino acid sequence of the complete protein. More preferably, the homology is at least 50%, even more preferably 75% across the whole of amino acid sequence of the protein or protein fragment. More preferably, homology is greater than 80% across the whole of the sequence. More preferably, homology is greater than 90% across the whole of the sequence. More preferably, homology is greater than 95% across the whole of the sequence.

It is envisaged that such molecules will be useful for the treatment of FSGS these molecules will bind specifically to CASK and will thus prevent cytoskeleton reorganization in podocyte induced by CASK and thus prevent proteinuria. As used herein, “binding specifically” means that the functionally equivalent analogue has high affinity for CASK but not for control proteins. Specific binding may be measured by a number of techniques such as ELISA, flow cytometry, western blotting, or immunoprecipitation. Preferably, the functionally equivalent analogue specifically binds to CASK at nanomolar or picomolar levels.

The polypeptides of the invention may be produced by any suitable means, as will be apparent to those of skill in the art. In order to produce sufficient amounts of hCD98 or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In one embodiment, the agent is an aptamerAptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

Another object of the invention is an inhibitor of hCD98 gene expression for use in the treatment of focal segmental glomerulosclerosis (FSGS) in a subject in need thereof.

An “inhibitor of expression” refers to a natural or synthetic compound that has a biological effect to inhibit the expression of a gene. Therefore, an “inhibitor of hCD98 gene expression” denotes a natural or synthetic compound that has a biological effect to inhibit the expression of hCD98 gene.

In a preferred embodiment of the invention, said inhibitor of hCD98 gene expression is a siRNA, an antisense oligonucleotide or a ribozyme.

Inhibitors of hCD98 gene expression for use in the present invention may be based on antisense oligonucleotide constructs. Anti-sense oligonucleotides, including anti-sense RNA molecules and anti-sense DNA molecules, would act to directly block the translation of hCD98 mRNA by binding thereto and thus preventing protein translation or increasing mRNA degradation, thus decreasing the level of hCD98, and thus activity, in a cell. For example, antisense oligonucleotides of at least about 15 bases and complementary to unique regions of the mRNA transcript sequence encoding hCD98 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of hCD98 gene expression for use in the present invention. hCD98 gene expression can be reduced by contacting the tumor, subject or cell with a small double stranded RNA (dsRNA), or a vector or construct causing the production of a small double stranded RNA, such that hCD98 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of hCD98 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of hCD98 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of hCD98 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing hCD98. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Chiffon, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Another object of the invention relates to a method for use in the treatment or prevention of FSGS comprising the administration of a therapeutically effective amount of at least one agent effective to inhibit the binding of CASK to hCD98 present on the surface of podocytes or inhibitor of hCD98 gene expression to a subject in need thereof.

By a “therapeutically effective amount” of the agent effective to inhibit the binding of CASK to hCD98 present on the surface of podocytes or inhibitor of hCD98 gene expression as above described is meant a sufficient amount of the agent effective to inhibit the binding of CASK to hCD98 present on the surface of podocytes or inhibitor of hCD98 gene expression to treat o prevent FSGS. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day.

Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The agent effective to inhibit the binding of CASK to hCD98 present on the surface of podocytes or inhibitor of hCD98 gene expression of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The agent effective to inhibit the binding of CASK to hCD98 present on the surface of podocytes or inhibitor of hCD98 gene expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifuCASK agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The agent effective to inhibit the binding of CASK to hCD98 present on the surface of podocytes or inhibitor of hCD98 gene expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Screening Methods of the Invention:

The present invention also relates to a method of screening a drug for the treatment or prevention of FSGS which comprises the step consisting of determining whether a candidate compound is efficient to inhibit the binding of CASK to hCD98 present on the surface of podocytes and ii) positively selecting the candidate compound that is efficient to inhibit the binding of CASK to hCD98 present on the surface of podocytes.

In a particular embodiment the method comprises the step consisting of:
a) immobilizing an CASK on a solid support;
b) contacting the immobilized CASK with sufficient detectable hCD98 to saturate all binding sites for the hCD98 on the immobilized CASK under conditions permitting binding of the hCD98 to the immobilized CASK so as to form a complex;
c) removing unbound hCD98;
d) contacting the complex with the compound; and
e) determining whether any hCD98 is displaced from the complex, wherein displacement of hCD98 from the complex indicates that the compound binds to CASK.

In another particular embodiment, the method comprises the step consisting of:
a) immobilizing a hCD98 on a solid support;
b) contacting the immobilized hCD98 with sufficient detectable CASK to saturate all binding sites for CASK on the immobilized hCD98 under conditions permitting binding of the immobilized hCD98 to CASK so as to form a complex;
c) removing unbound CASK;
d) contacting the complex with the compound;
e) determining whether any CASK is displaced from the complex, wherein displacement of CASK from the complex indicates that the compound binds to the hCD98.

In another particular embodiment, the method comprises the step consisting of:
(a) contacting an CASK with sufficient detectable hCD98 to saturate all binding sites for the hCD98 on CASK under conditions permitting binding of the hCD98 to CASK so as to form a complex;
(b) removing unbound hCD98;
(c) measuring the amount of hCD98 which is bound to CASK in the complex;
(d) contacting the complex with the compound so as to displace hCD98 from the complex;
(e) measuring the amount of hCD98 which is bound to the compound in the presence of the compound; and
(f) comparing the amount of hCD98 bound to CASK in step (e) with the amount measured in step (c), wherein a reduced amount measured in step (e) indicates that the compound binds to CASK.

In another particular embodiment, the method comprises the step consisting of:
(a) contacting an CASK with the compound and detectable hCD98 under conditions permitting binding of the hCD98 to CASK in the absence of the compound so as to form a complex;
(b) removing unbound hCD98;
(c) comparing the amount of detectable hCD98 which is bound to CASK in the complex in the presence of the compound with the amount of detectable hCD98 which binds to the compound in the absence of the compound; wherein a reduced amount of hCD98 measured in presence of the compound indicates that the compound binds to CASK or hCD98.

In the methods described herein, an entity may be made detectable by labeling it with a detectable marker. In one embodiment of the methods described herein, the detectable hCD98 is labeled with a detectable marker. In one embodiment of the methods described herein, the detectable CASK glycoprotein is labeled with a detectable marker. One skilled in the art would know various types of detectable markers. Such detectable markers include but are not limited to a radioactive, calorimetric, luminescent and fluorescent markers.

In one embodiment of the method described herein, the solid support is a microtiter plate well. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip.

In one embodiment of the above methods, the detectable molecule is labeled with a detectable marker. In another embodiment of the above methods, the detectable molecule is detected by contacting it with another compound which is both capable of binding the detectable molecule and is detectable. The detectable markers include those described above.

As used herein, the term “candidate compound” includes both protein and non-protein moieties. In one embodiment, the candidate compound is a small molecule. In another embodiment, the candidate compound is a protein. The protein may be, by way of example, an antibody directed against a portion of CASK. The candidate compound may be derived from a library of low molecular weight compounds or a library of extracts from plants or other organisms. In an embodiment, the agent is known. In a separate embodiment, the candidate compound is not previously known. The agents/compounds of the subject invention include but are not limited to compounds or molecular entities such as peptides, polypeptides, and other organic or inorganic molecules and combinations thereof.

The invention will be further illustrated by the following figures and examples. However, these examples and figures should not be interpreted in any way as limiting the scope of the present invention.

### EXAMPLE

### Cask, A Soluble Factor Involved in Focal Segmental Glomerulosclerosis

1—Soluble CASK is Associated with Recurrent FSGS

The main goal of this study consists in the identification and characterization of the soluble factor involved in the pathogenesis of FSGS. We are particularly interested to study its recurrence in allografts for diagnostic, prognostic and therapeutical purposes.

As mentioned above, plasmapheresis/immunoabsortion removes this molecule from serum improving drastically the symptomatology of FSGS patients. Based on these facts, we applied an approach of immunoabsortion in protein-A and further mass spectrometry MS analysis as initial starting point.

Serum from patients were collected and processed for MS. By electrophoresis (PAGE) it was noticed a well-differentiated band (˜100 kDa) which was digested by trypsin. After alkylation, the recovered peptides were analyzed and processed to mass spectrometry MS/MS in a nanoelectrospray LC-QTOF (liquid chromatography-quadrupole time-of-flight) spectrometer. After performance of such procedure, we have obtained 8 peptides, which 100% match to human CASK sequence.

Once identified this protein, we proceeded to verify its connection to the pathogenesis of FSGS. Hence, we have firstly checked the presence of CASK in serum from patients related to health controls. Tests were performed by dot-blot anti-CASK. The presence of CASK in serum was only found in patients. Identical experiments were accomplished by using anti-Nephrin done in the same patients. Results demonstrated the absence of Nephrin in serum of all patients. These data demonstrated that the serum origin of CASK is other than a contamination due to massive glomeruli destruction.

In order to get better information about the specificity of CASK in patients undergoing FSGS, protein-G immunodepletion followed by western anti-CASK were carry out. The presence of a 114-kDa band corresponding to the full-length isoform of CASK was clearly illustrated. Remarkably, the presence of this band is exclusive to HSGS patients but not in the health control.

CASK has been described as intermembrane protein; therefore its unexpected presence serum from FSGS patients must be a matter of extensive studies. One possibility is the existence of an aberrant mechanism of expression-secretion in blood cells. As a clue, we have found a high expression of CASK in malignant cell lines of lymphoid origin demonstrated the expression of CASK in Burkitt's lymphoma BL41 cells but not in RCC7 cells proceeding from a renal clear cell carcinoma. Likewise, CASK was also expressed by Jurkat leukemia T cells. Those data could suggest an extraglomerular origin of CASK in serum being generated by a potential secretion of blood cells.

2—Soluble CASK Induces Podocyte modifications, cell contact alteration and albuminuria.

To determine the implication of soluble CASK in the physiopathology of FSGS, we have tested the interaction of CASK with Podocytes.

For in vitro experiments in podocytes we have cloned and expressed recombinant CASK for in vitro experiences into pCDNA3.1+ and pTRC2, polyhistidin tagged.

The potential activity of recombinant CASK was tested in a podocyte cell line immortalized by SV40. We have explored a potential CASK-induced alteration over the structure of the membrane tight junctions by immunoflorescence anti-ZO-1. An apparent disappearance of ZO-1 from intercellular junctions of podocytes was shown. Similar results were found on distribution of intermembrane p-Cadherin. These results suggested a direct effect of CASK in the membrane organization and polarity of the cell.

Cytoskeleton organization is essential to the maintenance of cell polarity and to the distribution and stability of membrane proteins. Under this scenario, actin microfilaments play a critical role. Experiments performed in podocytes treated by recombinant CASK, revealed the expression of actin stress fibers, which are currently associated to many cellular functions, as morphological stability or adhesion.

To test the effect of soluble CASK on glomerular structure, recombinant CASK (60 μg/injection) or control protein (bovine serum albumin 60 μg/injection) have been injected IV 3 consecutive days to mice (3 mice per group). Mice have been sacrificed the day after the last injection and kidneys have been prepared to be analyzed by electron microscopy. In control group (left panels), Podocytes (P) exhibit typical foot processes (orange arrows) whereas in mice injected with CASK (right panels), podocytes show fusion of foot processes (red arrows).

We have tested the effect of the recombinant CASK on the maintenance of contact adhesion and the absence of protein translocation of a monolayer of podocytes cultured on filters. Mouse Albumin was added in the upper chamber and the media of the lower chamber was tested for the occurrence of mousse albumin by ELISA. The addition of Cask on the monolayer was associated with an increase translocation of mouse albumin.

We tested also the ability of a single injection of CASK to induce proteinuria in SCID mice. 20 μg of CASK or vehicle have been injected in 4 mice. The urines have been collected 24 hours later during a bladder squize.

We also injected CASK (20 μg/ml) (200 μl) IV in FVB mice (n=3) and urine have been collected in metabolic chambers for one day. Mouse albuminuria has been determined by ELISA (Bedhyl). Injection of CASK is associated with an increase of albuminuria (mean value+140%). After a single injection, proteinuria went down to the normal value and re-increase following a second injection of CASK (20 μm/ml) (200 μl).

A second set of in vivo experiment has been performed with 2 groups of mice receiving either vehicle (CTRL) or CASK (20 μg) IV. The proteinuria after CASK injection is significantly increase as compared to control animals.

3—Soluble CASK Bind to CD98 in Podocytes

We looked for a potential receptor of CASK on podocytes. Very recently, CD98, a membrane protein, has been described as a ligand for CASK in intestinal epithelium. CD98 exhibits a large extra-membrane region of ˜500 residues including a PDZ-binding domain. By IP analysis and after crosslinking of recombinant CASK with its receptor, we have shown that CD98 coprecipitated with CASK. We have cloned CD98 and expressed it in bacteria. We showed that soluble CASK bind to CD98 and can be depleted in the serum.

The results indicated that soluble CD98 can constitute a therapeutic tool to inhibit the interaction of CASK with its endogenous receptor.

4—CASK-Induced Signaling in Podocytes

As described in the introduction, intracellular CASK, as scaffolding protein, participates in the maintenance of polarized cell architecture by linking membrane proteins and further signaling to cytoskeleton. We observed that extracellular CASK is associated with the re-organization of the actin cytoskeleton. CD98 is supposed to be linked to the NFkB pathway. We have tested the activation of NFkB and its translocation of p65 to the nuclear structure. After 30 minutes p65 molecules was translocated to the nucleus. This correlates to the phosphorylation of ikB alpha This suggests that inhibition of NFkB would reduced the podocyte alterations induce by CASK.

Phosphorylation of ikB is associated with its degradation and the release in the cytoplasm of p65 and p50 NFkB which translocate into the nucleus. We therefore tested the nuclear translocation of P65 into the nucleus by immunofluorescence. Both of these effects (phosphorylation of ikB and nuclear translocation of NFkB) were not observed in cells incubated with anti CD98 mAB.

Incubation of podocytes with CASK also induces the activation of MAPK pathway. This pathway is also activated when cells are incubated with anti CD98 mAb.

Alltogether these results indicate that CASK induce the reorganisation of actin skeleton favor the redistribution of ZO-1 and activated NFkB pathway and MAPK pathway whereas the last is less specific tan the NFkB pathway.

We also determined whether CASK was associated with a modification of the ability of cells to move with a wound healing test. Confluent and differenciated podocytes were incubated or not with CASK. The size of the wound scare was determined 24 hours later. Extra cellular CASK is associated with a reduce ability of the cells to migrate.

### REFERENCES

Throughout this application, various references describe the state of the art to which this invention pertains. The disclosures of these references are hereby incorporated by reference into the present disclosure.

1. Crosson J T. “Focal segmental glomerulosclerosis and renal transplantation”. Transplant Proc. 39 (3):737-43. 2007

2. Daskalakis N, Winn M P. “Focal and segmental glomerulosclerosis”. Cell Mol Life Sci. 63 (21):2506-11. 2006

3. Asanuma K, Mundel P. “The role of podocytes in glomerular pathobiology”. Clin Exp Nephrol. 7 (4):255-9.2003

4. Lehtonen S, Lehtonen E, Kudlicka K, Holthöfer H, Farquhar MG. “Nephrin forms a complex with adherens junction proteins and CASK in podocytes and in Madin-Darby canine kidney cells expressing nephrin”. Am J Pathol. 165 (3):923-36. 2004

5. Yan Y, Vasudevan S, Nguyen H, Bork U, Sitaraman S, Merlin D. “Extracellular interaction between hCD98 and the PDZ class II domain of hCASK in intestinal epithelia”. J Membr Biol. 215 (1):15-26. 2007

1. A method for determining whether a subject is at risk of having or developing a focal segmental glomerulosclerosis (FSGS) or is at risk of recurrence of FSGS after renal transplantation comprising the step consisting of determining the level of calcium/calmodulin-dependent serine protein kinase (CASK) in a blood sample obtained from the subject.

2. The method according to claim 1 which further comprises a step consisting of comparing the determined level of CASK in the blood sample obtained from the subject with a reference level, wherein a difference between said determined level and said reference level is indicative whether said subject is at risk of having or developing a FSGS or is at risk of recurrence of FSGS after renal transplantation.

3-11. (canceled)

12. A method for determining the responsiveness of a subject suffering from a focal segmental glomerulosclerosis (FSGS) to a treatment, comprising the step consisting of determining the level of calcium/calmodulin-dependent serine protein kinase (CASK) in a blood sample obtained from the subject.

13. A method for the treatment or prevention of FSGS comprising the administration of a therapeutically effective amount of at least one agent effective to inhibit the binding of CASK to hCD98 present on the surface of podocytes or inhibitor of hCD98 gene expression to a subject in need thereof.

14. The method according to claim 13, wherein said agent is selected from the group consisting of antibodies, small organic molecules, and polypeptides.

15. The method according to claim 14, wherein said agent is an antibody or a fragment of an antibody having the ability to block the interaction between CASK and hCD98.

16. The method according to claim 15, wherein said agent is selected among antibodies or fragment of antibodies directed to all or a portion of the extracellular domain of hCD98.

17. The method according to claim 16, wherein said agent is an antibody binding to an epitope located in the class II PDZ-binding domain of hCD98.

18. The method according to claim 14, wherein said agent is an antibody or portion thereof capable of inhibiting the binding of hCD98 to CASK, wherein said antibody binds to an epitope located within a region of CASK, wherein said region of CASK binds to hCD98.

19. The method according to claim 15, wherein said agent is an antibody binding an epitope located in the PDZ class II domain of CASK.

20. The method according to claim 14, wherein said agent is a polypeptide comprising all or a portion of the extracellular domains of hCD98.

21. The method according to claim 20, wherein said agent is a polypeptide comprising the class II PDZ-binding domain of hCD98 or a portion thereof.

22. The method according to claim 13, wherein said inhibitor of hCD98 gene expression is a siRNA, an antisense oligonucleotide or a ribozyme.

23. A method of screening a drug for the treatment or prevention of FSGS which comprises the step consisting of determining whether a candidate compound is efficient to inhibit the binding of CASK to hCD98 present on the surface of podocytes and ii) positively selecting the candidate compound that is efficient to inhibit the binding of CASK to hCD98 present on the surface of podocytes.
